(19) United States
(12) Patent Application Publication
(10) Pub. No.: US 2010/0093101 A1
(43) Pub. Date: Apr. 15, 2010

(54) METHOD FOR DETERMINING THE CONTENT OF METALLIC ELEMENTS IN FISCHER-TROPSCH WAXES

(75) Inventors: Adrian Peter Darling, Sasolburg (ZA); Jacoba Petronella Coetzee, Sasolburg (ZA); Deborah Karen Yoell, Meyersdal (ZA)

Correspondence Address: KNOBBE MARTENS OLSON & BEAR LLP, 2040 MAIN STREET, FOURTEENTH FLOOR, IRVINE, CA, 92614, US

(73) Assignee: Sasol Technology (Pty) Ltd, Rosebank, Johannesburg (ZA)

(21) Appl. No.: 12/532,116

(22) PCT Filed: Mar. 19, 2008
(86) PCT No.: PCT/ZA08/00021

§ 371 Date: Dec. 8, 2009

(30) Foreign Application Priority Data
Mar. 20, 2007 (ZA) 2007/02300

(51) Int. Cl. G01N 33/20

(52) U.S. Cl. 436/76; 436/73; 436/79; 436/84

(57) ABSTRACT

The invention provides a method for determining the content of metallic elements in Fischer-Tropsch waxes by Inductively Coupled Plasma (ICP), wherein digestion of one or more samples of the waxes is carried out in an open vessel microwave digestion system. The invention further provides a sampling protocol for use with the method.

### FIELD OF THE INVENTION

The invention relates to a method for determining the content of metallic elements in Fischer-Tropsch waxes (FT waxes).

### BACKGROUND TO THE INVENTION

Metal species, and especially those of aluminium, present in hydrocarbon streams adversely affect the performance of hydroprocessing units, in particular affecting negatively the performance of the catalyst when processing synthetic feedstocks. These metal species tend to deposit on the hydrocracking catalyst with negative performance consequences. Therefore, and in particular, precise and accurate determination of the content of the metallic elements is vital to ensure the adequate performance over the expected lifetime of the catalyst.

It is an object of the developed method to assure conformance of the metal content of synthetic hydrocarbon streams to process specifications which are in the low ppm range.

A typical specification for elements like aluminium in heavy paraffin hydroprocessing feedstocks, such as the synthetic wax produced from a Fischer-Tropsch (FT) process, is set at <1 ppm. Therefore, accurate, quick and precise determination of the levels of elements like Na, K, Mg, Ca, Fe, Co and Al is of vital importance.

### SUMMARY OF THE INVENTION

According to a first aspect of the invention, there is provided a method for determining the content of metallic elements in Fischer Tropsch waxes by Inductively Coupled Plasma (ICP), wherein digestion of the waxes is carried out in an open vessel microwave digestion system.

The ICP may be selected from ICP-OES (Inductively Coupled Plasma Optical Emission Spectroscopy) and ICP-MS (Inductively Coupled Plasma Mass Spectroscopy).

The method is believed to be of great use in improving the catalytic hydroprocessing of synthetic feedstocks.

The method may be used to analyse for Na, K, Ca, Mg, Fe, Co and Al The method may be used for both organic and inorganic forms of Co and Al.

The open vessel microwave digestion system procedure may include a preparation procedure using sulphuric and nitric acids to digest the wax matrix enabling the trace element determination of metallic elements in FT wax.

However, other oxidizing acids such as perchloric acid, and oxidizing agents, such as hydrogen peroxide, may also be suitable for digesting the wax.

Digestion may be carried out for a period of under 90 minutes, typically under 60 minutes.

The method achieves a Level of Quantitation (LOQ) of below 1 ppm, typically below 0.6 ppm, for elements such as aluminium and cobalt.

A scrubber system may be used to reduce harmful vapours in the laboratory.

The sampling protocol was designed to enable homogeneous, representative wax samples to be taken on the plant. The wax may be sampled using metal, for example stainless steel, sampling containers and caps that have been steam cleaned to remove wax and contaminants including catalyst residue. The cleaned sampling containers and caps may be stored in a dust-free environment to prevent contamination prior to sampling.

The sampling containers may be 1 cm deep, or less, as this provides a homogenous wax sample. As a result accurate and repeatable results may be obtained.

Before the sample is drawn, the sampling lines, sample points and the sampling containers may be rinsed thoroughly with molten wax in order to get a representative sample. Once the molten wax sample is drawn, the containers may be capped and the sample left to fully congeal before being analysed. The caps may be used to protect the sample from contamination on the plant once the sample has been taken and during the solidification process. Once the wax has congealed, the sample may be transferred to a sealed bag and sent to the laboratory for analysis.

The sample may be digested by open vessel microwave using oxidising agents such as hydrogen peroxide, perchloric, sulphuric, and/or nitric acids and the elements of interest in the diluted solution then quantified by comparing the intensity at characteristic wavelengths to that of a series of standards using yttrium as internal standard.

The sample may be prepared by breaking up the wax ‘cake’ into representative pieces and weighing off 2-3 g of wax into the quartz digestion vessels. This may be followed by the addition of sulphuric acid (typically 15 ml). A procedure blank may be prepared using the same volume of sulphuric acid and is treated further in the same manner as the sample. The digestion vessels may then be loaded into the open vessel microwave digestion apparatus which is pre-programmed to add a total of approximately 50 ml of nitric acid during the course of the digestion. Sample digestion may take from 15 to 120 minutes, typically around 45 minutes, whereafter the samples and procedure blank are allowed to cool. To ensure that no sample is lost, the sides of the quartz digestion vessels may be washed down with deionised water. After mixing, the digested sample may be quantitatively transferred to a volumetric flask with deionised water and the internal standard is added. The digested sample may then be diluted to volume using deionised water.

The sample may now be ready for analysis by comparing the intensity at characteristic wavelengths to that of a series of standards using yttrium and scandium as internal standard.

The ICP instrument may be calibrated with a multi-element standard which includes yttrium or scandium as internal standards.

### DESCRIPTION OF EMBODIMENTS OF THE INVENTION AND COMPARATIVE DATA

A need arose to develop a suitable method for analysing metallic elements, in particular Al and Co, in a wax matrix that could achieve the accuracy and precision that was required. The challenge lay in the preparation of the sample as the wax matrix proved to be very difficult to digest.

A number of approaches to this analysis were considered and some tested for accuracy and precision. These included a combustion procedure (Method 1), a wet-ashing procedure (Method 2) and a closed-vessel microwave procedure (Method 3), all followed by quantification by ICP-OES.

Another methodology considered was an X-Ray (XRF) Fluorescence technique. In the XRF technique, during sample preparation, particulate form analyte would sink to the bottom of the sample giving false high results against homogeneous organometallic standards, especially with the low depth the light Al element is measured to.

In the three methods under consideration, recovery on both inorganic and organometallic forms of Al and Co was checked by spiking a sample with Co/alumina catalyst and Conostan oil-based organometallic standards. Precision was checked on a bulk sample prepared by melting a number of samples together. Developmental tests using spiked matrices is a general practice for difficult matrices.

TABLE 1
Accuracy and Precision data for Methods 1, 2 and 3

| | Method 1 | | Method 2 | | Method 3 | |
|---|---|---|---|---|---|---|
| | Precision data | | | | | |
| Parameter | Al | Co | Al | Co | Al | Co |
| Number of tests | 12 | 12 | 5 | 5 | 5 | 5 |
| Average result (ppm m/m) | 39 | 2 | 52 | 1.8 | 58 | <1.5 |
| Standard deviation (ppm m/m) | 2.7 | 0.3 | 3.0 | 0.11 | 12 | <1.5 |
| Coefficient of variance (%) | 6.9 | 14 | 5.8 | 6.5 | 20 | — |
| Accuracy data on organic form | | | | | | |
| Concentration in unspiked sample (ppm/m/m) | 1.1 | 0 | 1.1 | 0 | 58 | * |
| Added concentration (ppm m/m) | 42 | 48 | 56 | 20 | 59 | * |
| Found concentration (ppm m/m) | 30 | 39 | 54 | 18 | 101 | * |
| Recovery (%) | 72 | 82 | 95 | 89 | 86 | * |
| Accuracy data on inorganic form | | | | | | |
| Concentration in unspiked sample (ppm m/m) | 1.1 | 0 | 1.1 | 0 | * | * |
| Added concentration (ppm m/m) | 111 | 17 | 45 | 48 | * | * |
| Found concentration (ppm m/m) | 70 | 16 | 36 | 43 | * | * |
| Recovery (%) | 63 | 94 | 82 | 88 | * | * |

* not tested
** not determined

Method 1 uses a large sample size that will aid in overcoming sample heterogeneity, but shows poor recovery, possibly as a result of combusting the sample and in the process potential loss of volatile Al species. The aluminium results were found to be too low under these sample preparation conditions and this led to the evaluation of a wet-ashing procedure (Method 2). While this approach showed the best recoveries, it was not possible to achieve a limit of quantitation (LOQ) of <1 ppm and the sample preparation was very time consuming (4-6 hours). In addition, the large sample load posed a health and safety risk within the laboratory as large volumes of sulphuric and nitric acids were required to digest the wax matrix and the chance of contamination from the borosilicate glassware used in the digestion is high as the borosilicate glass can leach aluminium contributing to a high background and further compromising the LOQ. To improve the turn around time within the lab and to mitigate the health risks, a closed vessel microwave digestion procedure was attempted. This approach suffered as a result of the very small sample size that could be digested due to the tendency of the sample to react violently and uncontrollably under these conditions. Closed-vessel microwave digestion was unsuccessful as the sample size is limited due it being very reactive and exploding easily, the consequence of which is poor precision, and relatively poor LOQ's as insufficient sample size can be used. Recovery was not tested on inorganic form analyte due to the small sample size used.

A novel open-vessel microwave sample preparation procedure using sulphuric and nitric acids was thus developed to digest the wax matrix enabling the trace element determination of metals in FT wax. The digestion procedure was designed to be quick (digestion takes 45 minutes) and achieve good accuracy and precision. Using quartzware a LOQ of <0.23 ppm m/m can be achieved for Al based on a sample dilution of 2.5 g to 50 ml. A scrubber system reduces the harmful vapours in the laboratory.

A crucial part of the analysis is the sampling as homogeneous samples are required to achieve the required accuracy and repeatability. Sample heterogeneity is problematic with wax samples as the metal species/catalyst fines have a tendency to settle out as the wax solidifies; hence a new sampling technique was proposed to overcome this. The wax is sampled in such a manner as to minimise analyte discrimination during solidification of the wax. The benefit of this is improved sample homogeneity and better accuracy and precision, as described above.

Once an appropriate sample digestion procedure had been identified, it became necessary to focus on the quantitation of the elements in the wax. While doing this, it became apparent that the sulphuric acid matrix was adversely affecting the accuracy of the aluminium analysis due to transport interference (which was ±30% throughput relative to an aqueous standard for 40% H₂SO₄). This was corrected for using an internal standard typically yttrium or scandium. Both ionic and atomic emission lines of the internal standards were investigated and it was observed that the Al results were overcorrected for using ionic internal standard emission lines. This is illustrated in Table 2 using a 5 ppm m/v aqueous Al calibration standard.

TABLE 2
Effect of internal standards and sulphuric acid
concentration on a 5 ppm m/v aqueous Al standard

| | Al vs. Y | Al vs. Sc | Al vs. Y |
|---|---|---|---|
| % m/v | 414.284 nm | 361.383 nm | 414.284 nm |
| H₂SO₄ | ionic line | ionic line | atomic line |
| 0 | 5 | 5 | 5 |
| 10 | 5.15 | 5.15 | 4.93 |
| 15 | 5.19 | 5.12 | 4.94 |
| 20 | 5.34 | 5.28 | 4.91 |
| 40 | 5.86 | 5.85 | 4.81 |

From Table 2, it is evident that the yttrium atomic emission line is suitable as internal standard and as such is applied to the current microwave method. While this issue was minor relative to the digestion procedure, it is however still significant for the accurate quantification of aluminium.

A comparison of wet-ashing and microwave digestion techniques was conducted on several plant samples. The results are tabulated below.

TABLE 3
Comparison of techniques using ionic and
atomic internal standard emission lines

| | Wet-ashing | Wet-ashing | Microwave | Microwave |
|---|---|---|---|---|
| | Ionic Y | Atomic Y | Ionic Y | Atomic Y |
| Sample | line | line | line | line |
| 1 | 74.6 | 57.8 | 64.9 | 61.2 |
| 2 | 76.3 | 60.0 | 67.3 | 62.9 |
| 3 | 72.7 | 56.6 | 61.3 | 57.2 |

The difference in the results between the two techniques using the ionic Y line is attributed to the effect of the sulphuric acid concentration. As indicated in Table 2, the amount of sulphuric acid present adversely affects the quantification of aluminium. This effect is less noticeable in the microwave digestion as much less sulphuric acid is used for this digestion compared to the wet-ashing method; hence the overestimation of the aluminium content is less (Table 3). From the above results it is also evident that the wet-ashing and the microwave techniques both give very comparable results when using the atomic emission line for the Y internal standard.

Using the microwave technique excellent validation data can be achieved as indicated in Table 4.

TABLE 4
Validation data

Bulk homogenised sample

| Statistic | Al | Co |
|---|---|---|
| Average (ppm m/m) | 50.4 | 2.00 |
| Standard deviation (SD) | 0.97 | 0.25 |
| RSD (%) | 1.9 | 13 |
| Sample size | 10 | 5 |
| Recovery (%) | 97 | 105 |

In conclusion, the validated microwave method is applicable to the analysis of wax from the FT reactor and for the analysis of wax from the wax-treatment unit. The elements for which the method has been validated are Na, K, Ca, Mg, Fe, Co and Al. Elements that may also be included are Ti, Zr and Zn. The elements of interest in the digested diluted solution are quantified by comparing the intensity at characteristic wavelengths to that of a series of standards using yttrium as internal standard. The method is applicable in the range LOQ to 100 ppm m/m for each element of interest. The LOQ is achievable due to a large sample size used (10 times more than can be digested by closed-vessel microwave). The sensitivity of the method can be adjusted by altering the sample size or dilution of the sample or both. Developmental tests using spiked matrices have shown the accuracy to be 92-109% of the spiked concentration level of various elements for these matrices.

1-19. (canceled)

20. A method for determining a content of at least one metallic element in a Fischer-Tropsch wax, comprising:
digesting at least one sample of a Fischer-Tropsch wax in an open vessel microwave digestion system; and
determining a content of at least one metallic element in the sample by an analytical technique employing an inductively coupled plasma.

21. The method of claim 20, wherein the analytical technique is inductively coupled plasma optical emission spectroscopy or inductively coupled plasma mass spectroscopy.

22. The method of claim 20, wherein at least one metallic element is selected from the group consisting of Na, K, Ca, Mg, Fe, Co, and Al.

23. The method of claim 22, wherein at least one metallic element is in a form, selected from the group consisting of an organic form of Co, an inorganic form of Co, an organic form of Al, and an inorganic form of Al.

24. The method of claim 20, wherein digesting comprises using at least one oxidizing agent to digest the sample.

25. The method of claim 24, wherein at least one oxidizing agent is selected from the group consisting of hydrogen peroxide, sulphuric acid, nitric acid, perchloric acid, and combinations thereof.

26. The method of claim 20, wherein the sample is digested for a period of under 90 minutes.

27. The method of claim 20, wherein the sample is digested for a period of under 60 minutes.

28. The method of claim 20, wherein the metallic element is aluminium or cobalt, and wherein a level of quantitation of the metallic element of below 1 ppm is achieved.

29. The method of claim 20, wherein the metallic element is aluminium or cobalt, and wherein a level of quantitation of the metallic element of below 0.6 ppm is achieved.

30. The method of claim 20, further comprising, before digesting, obtaining a sample of a Fischer-Tropsh wax, wherein the Fischer-Tropsh wax is sampled using a metal sampling container and cap that have been steam cleaned to remove wax and contaminants including catalyst residue.

31. The method of claim 30, wherein the metal sampling container is a stainless steel sampling container.

32. The method of claim 30, wherein the sampling container is 1 cm or less deep.

33. The method of claim 30, further comprising, before obtaining a sample, rinsing thoroughly with molten wax all sampling lines, all sample points and all sampling containers to be used in obtaining the sample.

34. The method of claim 33 further comprising, after obtaining a sample, capping the sampling container and allowing the sample to fully congeal before being digested.

35. The method of claim 34, further comprising, after the sample is allowed to fully congeal, but before digestion, breaking up the congealed sample into a plurality of representative wax pieces, placing from 2 g to 3 g of the representative wax pieces in a quartz digestion vessel, and then adding sulphuric acid to the quartz digestion vessel.

36. The method of claim 35, further comprising preparing a procedure blank using a same volume of sulphuric acid that is treated further in a same manner as the sample.

37. The method of claim 36, further comprising loading the quartz digestion vessel containing representative wax pieces and sulfuric acid into the open vessel microwave digestion system, and adding a predetermined volume of nitric acid to the quartz digestion vessel during digestion, wherein the sample is digested for a period of from 15 minutes to 120 minutes.

38. The method of claim 37, further comprising, after digestion for a period of under 60 minutes, allowing the sample to cool, washing down sides of the quartz digestion vessel with deionized water, mixing the digested sample and the deionized water, quantitatively transferring the digested sample to a preselected volume volumetric flask with deionized water, adding an internal standard to the digested sample, and diluting the digested sample to a preselected volume using deionized water.

39. The method of claim 38, wherein determining a content of at least one metallic element in the sample comprises comparing intensity at characteristic wavelengths to that of a series of standards using yttrium or scandium as an internal standard.

40. The method of claim 20, wherein the analytical technique utilizes an inductively coupled plasma instrument calibrated with a multi-element standard which uses yttrium or scandium as an internal standard.
